(19) United States
(12) Patent Application Publication
(10) Pub. No.: US 2003/0078552 A1
(43) Pub. Date: Apr. 24, 2003

(54) Odor-controlling disposal container

(75) Inventors: Bruce Ernest Tepper, Cincinnati, OH (US); Angela Renee Ozias, Mason, OH (US); Ravindra Palitha Ranatunga, West Chester, OH (US); Ramon Andres Urteaga, West Chester, OH (US); Victor Nicholas Vega, Cincinnati, OH (US); Brandon Ellis Wise, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 09/871,923

(22) Filed: Jun. 1, 2001

(51) Int. Cl. A61F 13/15; A61F 5/44

(52) U.S. Cl. 604/333; 604/359

(57) ABSTRACT

This application discloses an odor-controlling container for use in disposing of wastes having an associated malodor, said container comprising container walls having inwardly-facing surfaces and outwardly-facing surfaces, said walls being joined so as to form an opening for receiving said wastes, said opening being fitted with sealing means, wherein an odor-controlling composition is distributed uniformly over the entirety of said inwardly-facing surfaces of said container.

### FIELD OF INVENTION

The present invention relates to an odor-controlling container for wastes having an unpleasant odor. The container is portable, flexible, self-sealable, and contains an odor-controlling composition deposited uniformly on the inwardly facing surfaces of the container walls. In certain embodiments the container may be comprised of a selectively-permeable barrier material.

### BACKGROUND OF THE INVENTION

Many wastes, when disposed as solids or liquids in or around the home and elsewhere, create unpleasant odors, attract undesirable insects and/or animals, and generally create an unpleasant environment in the immediate vicinity thereof. Malodors from bodily fluids and feces associated with absorbent articles and pets, putrefying organic materials associated with food wastes, combustion by-products associated with use of tobacco products, and the like can be difficult to control. There is, in addition, a common need for discrete, odor-controlled disposal of solid wastes when away from home for soiled absorbent articles like diapers, sanitary napkins, pantyliners, adult incontinence products, as well as for food and pet wastes. Disposal of such wastes in paper and/or plastic bags does little or nothing to alleviate the odor problems, hence, there is a need for portable disposal containers that control odors.

Some waste containers rely on sorbents to control malodors by sorption of gases. JP2000-141553A2 describes a deodorizing and odor removing bag wherein a deodorizing sheet containing activated carbon is mounted with adhesive onto an inwardly-facing surface of the bag and wherein an air-permeable material covers the deodorizing sheet which is further protected prior to use by a releasable barrier. WO 00/29311A1 describes a flexible bag for food waste disposal with liquid impervious walls, and both a moisture absorbent and an odor-neutralizing composition within the bag. The odor neutralizing composition can comprise cyclodextrin, activated carbon, baking soda, absorbent gelling material, zeolite, silica, and/or mixtures thereof, and may also include a chelant and/or antimicrobial agent. JP11014512A2 describes a feces sampling container with odor control from inclusion of a metal oxide and silica gel complex, distilled plant extract and graft copolymer deodorant. JP10304786A describes a sealable paper recovery/disposal bag for animal and/or pet feces, wherein the bag may contain activated carbon or other deodorizer, and wherein the bag is flushable in a toilet since it dissociates in water. DE4304715A1 describes a liquid-collecting, odor-controlling, kitchen garbage bag made of decomposing material, ideally paper, for compostable wastes, with activated carbon, zeolite and/or clay sorbent. U.S. Pat. No. 4,861,632 describes a multi-laminated bag with internal layers of moisture-absorbent paper towel sheets filled with activated carbon and/or silica gel to sorb odors.

Some odor-controlling products involve bag-like containers specifically designed for disposal of absorbent articles. JP11056903A describes a folded disposal bag attached to a diaper or sanitary napkin that is used to enclose the soiled absorbent article for disposal, wherein flavonoid and amphoteric surfactant deodorants are stored within the disposal bag. U.S. Pat. No. 4,349,104 describes a scented disposal bag for absorbent articles, which releases a deodorizing fragrance within the bag when sealing the bag by its drawstring. Single-use, scented, diaper disposal bags that are closed by tying together the bag handles are currently marketed in the United Kingdom in supermarkets and elsewhere as Nappy Sacks. Very similar products are manufactured by Barna Ltd., Naperville, Ill., and marketed in the U.S. under the brand names Barna in supermarkets, Equate in Wal Mart Stores and Sassy in Baby Super Stores. Single-use, scented diaper disposal bags that are closed with separate twist ties are distributed by Toys “R” Us, Paramus, N.J., and CVS Pharmacy, Inc., Woonsocket, R.I.

Some odor-controlling products rely on both sorbent and fragrance to control malodors. WO8910311A1 describes a biodegradable disposal bag for organic waste wherein a clay adsorbent is mixed with fragrance.

The most common reasons for failure of containers involving only sorption technology are: 1) in the absence of 100% sorption of malodors, many residual non-sorbed malodors at parts per billion (ppb) levels or lower are perceived as obnoxious by humans, and 2) containment of the odors in disposal containers is typically insufficient to maximize the efficacy of the sorbent.

Two common reasons for failure of containers involving fragrance technology are that the fragrance used is: 1) insufficient in quantity compared to the levels of malodor to be controlled and/or, 2) of inferior malodor masking ability.

Inventions involving both sorbent and fragrance technologies often fail due to their individual shortcomings, and because the sorbent tends to capture part of the fragrance thereby reducing both masking potential and sorbent efficacy. For example, U.S. Pat. No. 5,342,333 and U.S. Pat. No. 5,037,412 teach that use of a fragrance to mask malodor in absorbent articles can detract from the functionality of a dry malodor sorbent. WO 00/29311 teaches avoidance of using both fragrance and odor sorbents in odor-controlling food waste disposal bags.

Currently available containers that claim to control odors of disposed absorbent articles, like soiled diapers, fail to consistently control strong malodors during periods when such control is expected.

Accordingly, there is a continuing need for an economical, efficacious, easy-to-use, flexible, portable, odor-controlling container for discrete disposal of odoriferous wastes from bodily fluids and/or feces associated with absorbent articles and pets; putrefying organic materials associated with food wastes; combustion by-products associated with use of tobacco products; and the like. In particular, there is a continuing need for such containers to provide improved control of malodors generated by such wastes.

### SUMMARY OF THE INVENTION

Disclosed herein is an odor-controlling container for use in disposing of wastes having an associated malodor, said container comprising container walls having inwardly-facing surfaces and outwardly-facing surfaces, said walls being joined so as to form an opening for receiving said wastes, said opening being fitted with sealing means wherein an odor-controlling composition is distributed uniformly on the entirety of the inwardly-facing surfaces of the container. Optionally, the odor-controlling composition is also distributed on the entirety of the outwardly facing surfaces of the container.

In a preferred embodiment the container is a sealable, flexible, odor-controlling container. Such containers are useful for disposing of soiled absorbent articles, such as diapers, sanitary napkins, pantyliners, tampons, adult incontinence articles, and the like, and/or other wastes associated with a broad spectrum of odoriferous materials, such as animal (including human)_bodily fluids and feces, food wastes, combustion by-products from tobacco products, and the like, any of which may or may not contain reactive functional groups, wherein an odor-controlling composition is distributed substantively in a uniform manner to cover the entirety of the inwardly facing surfaces of the container walls.

It is preferred that the instant container can be closed and the closure sealed by localized interlocking of the container walls, folding over the container walls, localized joining of the container walls with an adhesive or cohesive sealant, and any combination thereof.

It is preferred that the odor-controlling container of the present invention comprise an odor controlling composition comprising activated carbon, and additional components selected from the group consisting of a crystalline carboxylic acid, a metal oxide, a zeolite, a fragrance, and mixtures thereof.

It is preferred that the instant container stand upright when empty to receive waste through its open end without use of separate external support.

The walls of the instant container comprise a film, nonwoven and/or laminate materials. The walls of the instant container can be either permeable or impermeable to moisture depending on the intended wastes to be disposed. In addition, it is preferable that the walls of the present invention are generally permeable to carbon dioxide, methane, and organic compounds having one to three carbons with one of the following functional groups: alcohol, aldehyde, ester, and/or ketone.

### DETAILED DESCRIPTION OF THE INVENTION

Definitions

By “odor” herein is meant any substance that can stimulate an olfactory response in a human; i.e., sense of smell.

By “malodor” herein is meant an odor that is generally considered obnoxious or nauseating by the general population, such as the odor of animal feces, vomitus and some putrefying food wastes.

By “bodily fluids” herein is meant any flowable material of human origin, including but not limited to blood, feces, lymph, menses, perspiration, urine and vomitus.

By “absorbent” herein is meant the general case of a liquid and/or solid material into which are taken in one or more other gaseous, liquid and/or solid materials.

By “adsorbent” herein is meant the general case of a liquid and/or solid material having a surface onto which a thin layer of one or more other gaseous, liquid and/or solid materials become adhered.

By “flexible” herein is meant, when referring to a container, having the ability to be collapsed and/or folded by hand, or when referring to a material, having a bending length in the machine direction and/or cross-machine direction less than about 190 mm (millimeters) as measured by the “Standard Test Method for Stiffness of Nonwoven Fabrics Using the Cantilever Test”, ASTM designation D 5732-95, or equivalent.

By “inwardly-facing surfaces” is meant the surfaces of the bag oriented toward the waste material contained therein.

BY “outwardly-facing surfaces” is meant the surfaces of the bag oriented away from the waste material container therein.

By “permeable” herein is meant having pores, gaps or other means through which fluids (gases and/or liquids) can pass. When referring to a liquid no force beyond gravity is necessary for the liquid to move across a liquid-permeable material once that material is saturated with that liquid. When referring to a gas no force beyond simple diffusion, the movement of molecules from higher to lower concentration, is necessary for the gas to move across a gas-permeable material once that material is saturated with that gas.

By “sorbent” herein is meant the general case for an absorbent material and/or an adsorbent material.

By “containment” herein is meant the confining within a structure of entities, such as gases, liquids and/or solid, the majority of those entities, for an unspecified period of time.

By “perfume raw material” or “PRM” is meant any individual organic or inorganic chemical compound which, when in a gaseous state, can potentially be perceived by the human olfactory sense.

By “fragrance” herein is meant any mixture or composition consisting of one or more perfume raw material(s) with or without one or more carrier solvent(s).

By “fragrance masking” herein is meant to hide or disguise the olfactory perception of an odor with a fragrance, wherein the perception of the masking fragrance intensity is generally perceived to be greater than the original odor intensity in the absence of the fragrance.

By “fragrance neutralization” herein is meant to alter or disguise the olfactory perception of an odor with a fragrance, wherein the perception of the neutralizing fragrance intensity is generally_perceived to be less than the original odor intensity in the absence of the fragrance.

Container

The container disclosed herein is intended for odor-controlling disposal of odoriferous wastes, such as soiled absorbent articles, animal bodily fluids and feces, food wastes, combustion by-products from tobacco products, and other wastes evolving primarily organic_gases generally considered unpleasant to smell. The malodor-producing materials are placed into the instant odor-controlling container, the container is sealed, and then the container is placed into a trash or other solid waste receptacle.

The container disclosed herein can be provided in any number of configurations, and is most conveniently in the form of a flexible pouch, sack or “bag” which has sufficient volume to contain the waste being disposed. Approximate suggested suitable bag internal volumes for the present invention for representative wastes to be disposed therein are: 3 to 4 liters (L) for baby diapers, 0.5 to 2 L for sanitary napkins, 0.25 to 1 L for pantyliners, 0.5 to 6 L for adult incontinence articles which vary widely for size and design, 0.5 to 2 L for pet (e.g., dog, cat) feces, 4 to 8 L for contents of kitty litter pans, 4 to 40 L for food wastes, and 0.25 to 2 L for combustion by-products from tobacco products.

The opening of the container should be of sufficient size to permit easy entry of the wastes to be disposed. Approximate suggested suitable cross-sectional areas for the opening of the present invention for representative wastes to be disposed therein are: 300 to 600 cm² for baby diapers, adult incontinence articles, sanitary napkins, pet (e.g., dog, cat) feces, and contents of kitty litter pans; 150 to 300 cm² for pantyliners and combustion by-products from tobacco products, and up to 3000 cm² for food wastes.

The container can be constructed using any number and combination of materials, including film, nonwoven and/or laminate materials. Typical films are polyethylene, polypropylene, polyvinyl alcohol, polyvinyl acetate and mixtures thereof. Films can be single or multiple (either co-extruded or laminated) layers of cast or blown type. Nonwovens can be spunbonded, carded, airlaid with either calendar or air-through bonding, needle punched, melt blown, or spun lace. Laminates can be any combination of nonwovens and films joined by direct extrusion coating, adhesive, thermal bonding, pressure bonding, or ultrasonic bonding. Laminates can also be any combination of two or more nonwovens joined by adhesive, thermal bonding, pressure bonding, or ultrasonic bonding.

The materials used to construct the container can be either permeable or impermeable to liquids, depending on whether or not the wastes to be contained in specific embodiments of the present invention pose a leakage problem. For example, wastes to be disposed that are essentially dry, such as soiled absorbent articles containing super sorbents therein or cigarette ash, are less likely to need liquid impermeable walls because these wastes are unlikely to pose a leakage problem. Some other wastes, such as fish, meat, poultry or liquid food wastes, bodily fluids and pet feces, are more likely to pose a leakage problem such that liquid impermeable walls would be necessary or preferable.

It is preferable that the container wall materials do not form an impervious barrier to low molecular weight (MW) gases, such as carbon dioxide, methane, and organic compounds having one to three carbons with at least one of the following functional groups: alcohol, aldehyde, ester, and/or ketone. These gases are either odorless or not particularly malodorous, and typically represent the bulk of gases arising from the microbial degradation of the wastes being disposed and/or of secondary reaction products thereof. It is beneficial to dissipate, rather than to accumulate, these gases for both safety and efficacy reasons.

The container can be constructed as a seamless or joined product. Commonly known methods for forming containers from thermoplastic material include center-folding a flat web and using a cut seal on the lateral sides to form bags. When the present invention is not constructed in a seamless process, it is preferable that the joining regions of the container walls of the present invention are of sufficient strength to maintain integrity during the loading of wastes and thereafter during disposal. When present, joined regions should have the same or superior impermeability properties as the container walls themselves.

It is preferred that the container be constructed such that it can be expanded from a collapsed, compacted and/or folded state and made to stand freely without use of external supports, such as a stand or brace, in an open position to receive wastes. To achieve this end the walls of the container can be constructed in a variety of ways, for example with side or bottom gussets, accordion-folds, metal wires incorporated into the container walls, thickened or rigid regions of polymeric materials within the container walls, and any combination thereof.

Sealing Means

Sealing the container can be achieved in a variety of ways. The sealing means, in addition to helping provide for sanitary disposal of wastes, provides sufficient containment within the container of the odors evolving from the disposed wastes such that adequate time exists for the malodors to interact with components of the odor-controlling composition. A variety of closure mechanisms are possible to achieve this end. Practical and economical methods are preferred to effect a closure for the container, including interlocking of the container walls, folding over the container walls, localized joining of the container walls with an adhesive or cohesive sealant, and any combination thereof.

An interlocking seal for the container walls can be formed from the container walls themselves or by the addition of commonly-available closures added onto the container walls in the region of its opening. Examples of add-on closures include a tongue in groove seal like ZipPak® or slider seal like SliderGrip™, both by Minigrip®, and a hook and loop fastener like VELCRO®.

A seal produced by folding over opposing container walls can be formed by holding together the opposing walls in the region of the container opening and folding these over upon themselves, preferably at least twice. Alternatively, an extension of one of the container walls or a “flap” region can be folded over the container opening to effect a closure. The folded closure can be maintained by a variety of methods common in the art, including creasing of the container material during manufacture and/or by the user during use, and/or by securing the folded-over container walls in place by bending stiffened tabs incorporated into the container in the vicinity of its opening, and/or by an application of an adhesive of the types discussed below, and/or a hook and loop fastener. Other folding methods to effect a suitable closure can be envisioned, including those described next.

A variety of adhesives can be used to achieve satisfactory closure, which minimize odor loss from within the container. The seal can be formed using a pressure activated (pressure sensitive) adhesive or by an adhesive activated by removal of a protective strip over the adhesive, or both. The sealing means formed by use of adhesives can involve adhering the inwardly-facing surfaces of the container to one another, the outwardly-facing surfaces of the container to one another by folding both into the container, an inwardly-facing surface of one container wall to the opposing outwardly-facing surface of the container by folding some of the container wall into the container, the inwardly-facing surface of a flap or extension of one of the container walls to the outwardly-facing surface of the opposing container wall, or the outwardly-facing surface of one container wall to the opposing outwardly-facing surface of the container wall after the two walls have been folded upon each other. In addition, other folding methods can be envisioned that would effect a continuous seal of the container opening using adhesives.

Leakage of odors through the container closure can be reduced by lining the regions of interlocking and/or folded closures with an odor-controlling composition due to increased opportunity for interactions between the gases migrating through the closure and the odor-controlling composition. However, when effective levels of an odor-controlling composition are included in an adhesive closure the result is typically reduced efficacy of the adhesive thereby requiring more adhesive and/or a larger area of adhesive contact to effect sealing.

Odor-Controlling Composition

The odor-controlling composition of the present invention can be composed in any number of ways. In the present invention, the bulk of the malodors are intended to be controlled when they exist in a gaseous state rather than when they are in a pre-volatile state, such as when they are contained and/or dissolved within a liquid matrix.

Suitable materials for use in the composition are, at least partially, defined by the predominant malodor(s) to be sorbed. For example, the predominant malodors from fresh fish waste and fresh urine are amines. As microbial metabolism progresses over time during disposal, the nature of the malodors changes such that the predominant malodors in fish and urine change from amines to ammonia and thiols. In wastes like feces and menses the general tendency is for the predominant malodors from “young” samples to be organic acids, alcohols, amines and disulfides whereas the predominant malodors of “aged” samples that have undergone further anaerobic microbial degradation tend to be esters, ketones and thiols. However, malodors vary among such samples, as well as within samples over time, making it difficult to predict what malodors might be present for the whole class of wastes. In addition, some of the malodors such as skatole and hexanoic (caproic) acid appear to predominate from a sensory perspective because the human nose is more responsive to their presence rather than because their absolute concentrations are high when measured analytically. As mentioned earlier, the container walls are preferably permeable to non-malodorous low molecular weight gases, and impermeable to the majority of malodors, which have greater than three carbons.

Due to the inherent variability of the malodors to be managed from different wastes, it is preferable to use a combination of components in the odor-controlling compositions, though a single-component odor-controlling composition can provide adequate performance when applied appropriately. For example, activated carbon is a broad-spectrum adsorbent well known in the art. Activated carbon used alone can be efficacious for controlling a variety of malodorous gases, particularly malodorous organic compounds as the number of carbons in those compounds increases beyond three. Activated carbon is particularly effective when those malodors can be prevented from dispersing into the general environment by sustained containment which permits sufficient time for adsorption of malodors to take place, as discussed previously under “Sealing Means”. Activated carbon, however, is a relatively ineffective sorbent for some malodors common to some bodily fluids and food wastes, such as ammonia and very low molecular weight amines and thiols.

The activated carbon for the present invention needs to be optimized such that adequate capacity, adsorption kinetics and malodor retention are balanced amongst one another. For example, activated carbon granules below about 40 μm diameter tend to have relatively poor retention of adsorbed malodors. Activated carbon granules above about 250 μm diameter tend to have relatively good retention of adsorbed malodors, but relatively slower short-term (up to four hour) kinetics and some unusable sorption capacity deep within the granules. For the odor-controlling composition of the present invention activated carbon granules between 40 μm and 250 μm diameter are preferred, and between 80 μm and 125 μm more preferred. A representative activated carbon granule for use in the odor-controlling composition of the present invention is RGC 80×325 mesh size from Westvaco Corporation of Charleston, S.C. U.S.A. Activated carbon fibers and cloth, such as FM4/250 from Calgon Carbon Corporation of Pittsburgh, Pa. U.S.A., are also preferred for their sorption kinetics, capacity and malodor retention, but their cost can be prohibitive for commercially-viable embodiments of the present invention.

Other crystalline materials are also useful for the odor-controlling composition of the present invention. Mono-, di- and tri-carboxylic acids such as adipic, citric, maleic, malic and malonic acid, and polymeric acids such as alginic and polyacrylic acid, can mitigate odoriferous compounds by chemical neutralization. Such acids are effective for malodors that have a polar nitrogen, such as ammonia and organic amines from fish, urine and menses wastes. For the odor-controlling composition of the present invention, polyacrylic acid is preferred, alginic acid is more preferred, and citric acid is most preferred. In embodiments of the odor-controlling composition containing activated carbon and citric acid, the ratio of activated carbon to citric acid is from 1 to 10 to 10 to 1 by weight.

Metal oxides, such as aluminum, copper, magnesium, manganese, silica, titanium and zinc oxides, can be used to control odors that have a polar nitrogen, polar carboxylic acid group and/or a polar thiol group. For the odor-controlling composition of the present invention zinc oxide is preferred and silica dioxide is more preferred. Some zeolites of aluminosilicate composition have similar odor controlling properties.

For use in the odor-controlling composition disclosed herein, crystalline acid and metal oxide granules between 40 μm and 250 μm diameter are preferred, and between 80 μm and 125 μm more preferred. When acid and/or metal oxide granules are used in combination with activated carbon granules for the present invention, it is preferred that the acid and metal oxide granules should be on average at least half the diameter of the activated carbon granules and not greater than twice the diameter of the activated carbon granules.

Fragrance neutralization and/or fragrance masking alone is rarely effective against strong malodors such as from feces, putrefying fish and vomitus, but can be effective when used in conjunction with other odor-controlling technologies that substantially reduce or eliminate some of the most offensive malodors. For a fragrance to be most effective as part of the odor-controlling composition of the present invention, interference with the activity and/or capacity of sorbents and other components of the odor-controlling composition being used should be minimal. It is preferable that any fragrance used is more persistent, lingering or “durable” than the malodors being controlled. It is more preferable that the fragrance being used in the present invention be of the fragrance neutralization type.

It is preferable that the fragrance component of the odor-controlling composition is comprised of one to twelve perfume raw materials (PRMs), more preferably of two to six PRMs, most preferably of three to four PRMs, wherein the PRMs are chosen from among aliphatic, aromatic, polycyclic and heterocyclic chemicals, and any combination thereof. The fragrance component of the odor-controlling composition can be premixed with other components of the composition or applied separately, for example, by spraying.

As disclosed herein, the odor-controlling composition is distributed on the entire inwardly-facing surface of the container walls, wherein the preferred distribution is substantially uniform, and a more preferred distribution is uniform with high-density, wherein high-density implies that the distance between any two neighboring particles of the odor-controlling substance on the surface of the container walls is, on average, no greater than the diameter of the smaller of the two particles. Given that the container walls of the present invention will be permeable to many malodors, the denser the distribution of the odor-controlling composition, the more likely that malodors will have physical contact with the odor-controlling composition and be captured prior to migrating out of the container, for example, through the container walls. In addition, a uniform and denser distribution creates the potential for optimal efficacy of the odor-controlling composition by providing the greatest available surface area for sorption and/or chemical neutralization to take place.

The odor-controlling composition may be adhered directly to the container walls of the present invention or entrapped within the container walls by any number of means. In addition, adhesion of the odor-controlling substance can be achieved with or without the use of a binder or adhesive. For example, it is convenient to apply the odor-controlling composition to a sheet material, prior to its assembly into the container of the present invention, by various printing processes. U.S. Pat. No. 5,693,385 and U.S. Pat. No. 5,540,916 provide examples of such printing methods to adhere activated carbon to the inner surface of paperboard for packaging. Yet another method is to apply the odor-controlling composition to the container walls prior to or during the making of the container as part of the extrusion process wherein the odor-controlling composition is dispensed onto what will become the inwardly-facing surfaces of the wall material post extrusion while the container material is still tacky and prior to finished roll winding or the container-making operation if the processes are linked sequentially. It is also convenient to apply the odor-controlling composition to the container walls prior to or during the making of the container by a spray-on process to distribute the adhesive binder onto the inwardly-facing surface of the container wall material upon which the odor-controlling composition can be distributed and adhered. U.S. Pat. No. 2,690,415 describes such a spray-on adhesive method to adhere activated carbon to an odor-controlling fluid permeable sheet material. Yet another method is to imbed the odor-controlling composition in a non-woven or fibrous matrix that can be applied as the inner surface of a laminate wall material. U.S. Pat. No. 4,289,513 describes an activated carbon sorption paper that could be used for this purpose. Similar processes can be used for film, non-woven, and laminate wall materials.

Both the inwardly-facing and the outwardly-facing surfaces of the present invention can comprise the odor-controlling composition. For example, the entire container wall can be comprised of an odor-controlling composition when a moisture permeable container is acceptable, as is the case for constructing the present invention from activated carbon cloth. Yet another example is to comprise the container wall of a laminate material wherein the inner most (inwardly-facing) layer is comprised of a sorbent material applied to or contained within it, the outermost layer is comprised of a fragrance-impregnated film, and wherein the two layers are kept separate by one or more laminate layers.

For materials used to construct the present invention the odor-controlling composition basis weight of container wall material is less than about 250 g/m², preferred is less than about 50 g/m², more preferred is less than about 25 g/m², and most preferred is less than about 5 g/m².

### EXAMPLES

### Example 1

An odor-controlling bag with moisture-permeable, gas-permeable walls for the disposal of soiled diapers was constructed from Calgon Carbon activated carbon cloth (ACC) FM4/250 faced on the outwardly-facing surface only or on both the outwardly-facing and inwardly-facing surfaces with a spun-bonded polyethylene nonwoven. The bag was formed by off-center-folding the surface of said ACC material, which will become the inwardly facing surface, upon itself such that about 60 mm of one end of the outwardly-facing surface was uncomplemented to later form a closure flap region, whereupon the lateral sides were stitched together or joined by adhesive except for the flap region, after which the bag was turned inside-out such that the inwardly-facing surface of said ACC material became the inside surface of the bag. Said ACC bag had an outer finished width of about 250 mm, an outer finished total length of about 260 mm of which about 60 mm of the length was a fold-over closure flap extending the full width of the bag. A hook and loop type fastener (VELCRO®) can be attached to the inwardly-facing surface of the fold-over flap and to the outwardly-facing surface of the opposing bag side, such that it can form a closure for said bag. An adhesive tape strip with protective release paper, such as 3M™ Double Coated Tape Series 300 Product Number 444, was affixed to the outwardly-facing surface of the bag about 5 mm below and parallel to the bag opening for the entire width of the bag on the side opposite the flap region.

### Example 2

An odor-controlling bag with moisture-impermeable, gas-permeable walls for the disposal of soiled diapers, catamenials, incontinence pads, and the like was constructed from a Westvaco activated carbon film (ACF) lot# 106R01, the polyethylene film being about 1 mil thick and_having an inwardly-facing and outwardly-facing surface and upon the inwardly-facing surface is printed and adhered about 3 g/m² Westvaco RGC 80×325 mesh activated carbon granules. A bag of finished size about 250 mm width and about 260 mm length of which about 60 mm of the open end of the bag was an uncomplemented flap region as in Example 1. The bag was formed by center-folding the outwardly-facing surface of said ACF material upon itself and using a cut heat seal on the lateral sides after which the bag was turned inside-out such that the inwardly-facing surface of said ACF material becomes the inwardly-facing surface of the bag. An adhesive tape strip with protective release paper, such as 3M™ Double Coated Tape Series 300 Product Number 444, was affixed to the outwardly-facing surface of the bag about 10 mm below and parallel to the bag opening for the entire width of the bag on the side opposite the flap region. The bag was sealed during its use by holding together the sides of the bag in the region of the opening, removing the protective release paper from the adhesive tape strip, folding the flap over the bag opening and adhering it to the adhesive tape strip such that the adhesive tape strip contacts the inwardly-facing surface of the flap region and forms a sealed closure when pressure was applied.

### Example 3

A bag of similar construction as in Example 2 of finished size about 250 mm width and about 240 mm length wherein the flap region consists of a fold over region wherein both bag sides extend to the opening. The adhesive tape is affixed to the outwardly-facing surface of one side of the bag about 5 mm below and parallel to the bag opening. The bag is sealed during its use by holding together the sides of the bag in the region of the opening, removing the protective release paper from the adhesive tape strip, folding the width of the bag in the opening region over 180 degrees at or slightly below where the adhesive strip ends, about 30 mm below and parallel to the opening, in a direction opposite the side bearing the adhesive strip, and folding the width of the bag over once more in the same direction as the first fold such that the adhesive strip comes into contact with the outwardly-facing surface of the opposite wall of the bag and adhering the adhesive tape strip to that side by applying pressure along the region where contact is made between the adhesive strip and the opposing outwardly-facing bag wall.

### Example 4

An odor-controlling bag similar to that described in Examples 2 and 3 except the material used to construct the bag is comprised of polyvinyl alcohol film having inwardly-facing and outwardly-facing surfaces and upon the inwardly-facing surfaces is deposited and adhered to the hot film, immediately after extrusion and prior to finish winding, about 5 g/m² of an odor-controlling composition, said composition comprising 60 wt % 120×230 mesh activated carbon granules, 20 wt % 100×325 mesh crystalline citric acid, and 20 wt % 100×325 mesh silica dioxide, and wherein a SliderGrip™ is heat-sealed around the region of the bag opening to effect a bag seal.

### Example 5

An odor-controlling bag for the disposal of pet feces, kitty litter, and/or food wastes similar to that described in Examples 2 and 3 except the finished bag size is about 250 mm width and 400 mm length, wherein the material used to construct the bag is comprised of a laminate material, the outwardly-facing laminate being polyethylene film and the inwardly-facing laminate being a spun-bonded polyethylene non-woven onto the inwardly-facing surface of which is embedded 2 g/m² of an odor-controlling composition, said composition comprising 60 wt % 120×230 mesh activated carbon granules, 20 wt % 100×325 mesh crystalline citric acid, and 20 wt % fragrance composition, and wherein a “tongue in groove” seal such as a ZipPak® is heat-sealed around the region of the bag opening to effect a seal.

### Example 6

An odor-controlling bag for the disposal of tampons and/or pantyliners similar to that described in Examples 2 and 3 except the finished bag size is about 125 mm width and 75 mm length.

### Example 7

An odor-controlling bag similar to that described in Examples 2 and 3 except the bag is constructed with a side gussets and a 2 mil film is used such that the bag can stand upright with an open end up by extending the gussets.

Other variations and embodiments can be envisioned based on the Summary of the Invention.

### TEST METHODS

Closed-container Odor Control Test: The Closed-container method involves sensory analysis by trained graders to evaluate the ability of deodorant compositions, such as sorbents, fragrances, and combinations thereof, to manage a variety of malodors from, for example, urine- and/or feces-soiled diapers. For the purpose of evaluating efficacy of odor-controlling diaper disposal bags this test was practiced as follows. Diapers freshly soiled with both feces and urine were collected from daycare centers and individual residences. Clean (negative control) or soiled diapers with and without odor-controlling compositions were placed randomly and individually into polyethylene containers of about 4L capacity with tight-fitting lids that were removed only during sensory evaluations. In the case of odor-controlling bags, diapers soiled with both urine and feces were placed individually into an odor-controlling bag which was then placed into a container and the lid affixed. Laboratory odor graders and/or panelists previously trained to distinguish different levels of odor intensity and/or odor qualities evaluate each opened container at prescribed time intervals in sensory chambers. Sensory graders were trained to use a 0 to 8 scale for diaper malodors and fragrance intensity, where 0 means no odor is perceived and 8 is the most intense odor. The containers were stored at room temperature (ca. 21° C.) until all observations were made, typically after 1 day and after 3 days.

Closed-room Odor Control Test: The “Closed-room” method involves sensory analysis by untrained panelists known to be familiar with the odors of interest, for example parents who routinely change soiled diapers, and/or trained graders to evaluate the ability of deodorant compositions, such as sorbents, fragrances, and combinations thereof, to manage a variety of malodors from, for example, urine- and/or feces-soiled diapers. For the purpose of evaluating efficacy of odor-controlling diaper disposal bags this test was practiced as follows. Freshly soiled diapers were collected from daycare centers and from individual residences, keeping separate those diapers that contained only urine from those that contained feces. For each treatment two diapers soiled with both urine and feces and five diapers soiled with urine only chosen randomly were placed into a new Cosco diaper pail (model 09-108 WHO), the diaper pail lid was closed and each diaper pail was placed on a three-foot high table in an individual clean olfactory sensory room of about 800 cubic feet volume and the room closed for about 15 hours equilibration at room temperature (ca. 21° C.). Except for the positive control the feces-soiled diapers were placed individually into odor-controlling bags; urine-soiled diapers were added to all the diaper pails without an odor-controlling bag. A previously-unused and empty diaper pail served as the negative control. The positive control pail had the full complement of soiled diapers, none of which were placed in odor-controlling bags. After equilibration, five to seven panelists and one to three trained graders entered each room together and evaluated the diaper pail odor with the lid opened. Odor intensities were judged on the same 0 to 8 scale used for the Closed-container test.

Closed-container Test Results: Results in Table 1 demonstrate that both a moisture- and gas-permeable odor-controlling bag of similar contraction to Example 1 and a moisture-impermeable bag of similar construction to Example 2 significantly (p<0.01) reduced the perceived malodors from feces-soiled diapers. Two bags with no odor-controlling compositions, a standard plastic grocery bag and a food storage bag had no effect on perceived malodors. Neither did a fragranced diaper disposal odor control bag manufactured by Barna Ltd., Naperville, Ill.

Closed-room Test Results: Results in Table 2 demonstrate that both a moisture- and gas-permeable odor-controlling bag of similar construction to Example 1 and a moisture-impermeable bag of similar construction to Example 2 significantly (p<0.01) reduced the perceived malodors from feces-soiled diapers. Standard plastic grocery bags had no effect on perceived malodors. Food storage bags also significantly (p<0.01) reduced the perceived malodors, but perceived malodor from these bags was significantly (p<0.01) greater than from both background (clean diaper pail negative control) and either of the odor-controlling bags similar in construction to Examples 1 and 2.

TABLE 1
Malodor intensity over time in closed-container test

| | Number | Day 1 | Day 1 Malodor^{b} | Day 3 | Day 3 Malodor^{b} |
|---|---|---|---|---|---|
| Treatment tested | tested | N^{a} | (mean ± s.e.) | N^{a} | (mean ± s.e.) |
| Control^{c} (soiled diapers) | 3 | 6 | 7.2 ± 0.2 | 6 | 7.5 ± 0.2 |
| ACC Bag^{d} | 7 | 13 | 0.5 ± 0.2* | 17 | 1.6 ± 0.4* |
| Westvaco AC Film Bag^{e} | 4 | 10 | 4.8 ± 0.4* | 9 | 6.4 ± 0.4* |
| Plastic Grocery Bags^{f} | 1 | 2 | 7.5 ± 0.5 | 2 | 8.0 ± 0.0 |
| Commercial OC Bags^{g} | 3 | 6 | 6.0 ± 0.4 | 6 | 7.0 ± 0.2 |
| Freezer Ziploc Glad Bags^{h} | 3 | 6 | 7.0 ± 0.0 | 6 | 7.8 ± 0.2 |

TABLE 2
Malodor intensity of opened diaper pail in closed-room test

| | Number | | Pail Malodor^{b} |
|---|---|---|---|
| Treatment tested | tested | N^{a} | (mean ± s.e.) |
| Control^{c} (soiled diapers) | 20 | 138 | 5.9 ± 0.2 |
| Empty (clean) diaper pail | 1 | 7 | 1.1 ± 0.3* |
| ACC Bag^{d} | 7 | 48 | 1.2 ± 0.2* |
| Westvaco AC Film Bag^{e} | 2 | 13 | 1.3 ± 0.4* |
| Plastic Grocery Bags^{f} | 2 | 14 | 6.6 ± 0.3 |
| Freezer Ziploc Glad Bags^{h} | 2 | 14 | 3.9 ± 0.4* |

Notes for Tables 1 and 2:
*Significantly different (p < 0.001) from control by Student t-test.
^{a}N = number of individual evaluations by odor graders among tests.
^{b}Malodor intensity of opened container using 0 to 8 scale, mean ± standard error (s.e.), where 0 = no perciptible odor and 8 = highest possible odor intensity.
^{c}Diapers soiled with both feces and urine with no odor control treatment.
^{d}Soiled diaper containing feces and urine in Example 1 bag made of Calogon Carbon FM4/250 activated carbon cloth.
^{e}Soiled diaper containing feces and urine in Example 2 bag made of Westvaco film with print-on activated carbon granules, except the lateral sides of the bag were taped closed instead of heat sealed.
^{f}Soiled diaper containing feces and urine in polethylene grocery bag sealed by typing handles.
^{g}Soiled diaper containing feces and urine in fragranced odor control (OC) diaper disposal bag manufactured by Barna and scaled by typing handles.
^{h}Soiled diaper containing feces and urine in marketed one-gallon Glad ™ polyethylene freezer-type bag and Ziploc ® seal.

What is claimed is:

1. An odor-controlling container for use in disposing of wastes having an associated malodor, said container comprising container walls having inwardly-facing surfaces and outwardly-facing surfaces, said walls being joined so as to form an opening for receiving said wastes, said opening being fitted with sealing means, wherein an odor-controlling composition is distributed uniformly over the entirety of said inwardly-facing surfaces of said container.

2. The odor-controlling container of claim 1, wherein said odor-controlling composition is additionally distributed uniformly over the entirety of the outwardly-facing surfaces.

3. The odor-controlling material of claims 1 or 2 wherein the odor-controlling composition comprises activated carbon cloth.

4. The odor-controlling container of claim 1 wherein the container walls are moisture impermeable.

5. The odor-controlling container of claim 1 wherein the odor-controlling composition comprises activated carbon and additionally comprises a component selected from the group consisting of metal oxides, crystalline organic carboxylic acid, fragrance, or mixtures thereof.

6. The odor-controlling composition of claim 5 wherein the crystalline organic carboxylic acid is citric acid.

7. The odor-controlling container of claim 1 wherein the container is flexible.

8. The odor-controlling container of claim 7 having an open position and a closed position wherein the container can stand upright in the open position, when empty or partially filled, without external support.

9. The odor-controlling container of claim 8 wherein the container is a gusseted bag.

10. The odor-controlling container of claim 1 wherein the container walls are comprised of a selectively-permeable barrier.

11. The odor-controlling container of claim 10 wherein the selectively-permeable barrier is generally permeable to carbon dioxide, methane, and organic compounds having one to three carbons said organic compounds having at least one of the following functional groups: acid, alcohol, aldehyde, and/or ketone.

12. The odor-controlling container of claim 1 wherein the sealing means is a physical seal.

13. The odor-controlling container of claim 12 wherein the physical seal is selected from the group consisting of an adhesive seal, a cohesive seal, a hook and loop fastener, a folded configuration, a tongue and groove seal, a sliding seal, and a zipper.

14. The odor-controlling container of claim 13 wherein the adhesive is pressure sensitive.

15. The odor-controlling container of claim 1 wherein the container walls are selected from the group consisting of: film, nonwoven or laminate materials, and combinations thereof.

16. The odor controlling container of claim 1 wherein the odor-controlling composition is uniformly distributed and adhered onto the inner surface of the container walls by a method selected from the group consisting of adhesive bonding, thermal bonding, pressure bonding, ultrasonic bonding, physical entanglement with fibers, printing and combinations thereof.

17. The odor-controlling container of claim 5 wherein the odor-controlling composition comprises fragrance comprising one or more perfume raw materials selected from the group consisting of
a) linear aliphatic and/or branched aliphatic compounds,
b) monoaromatic, polyaromatic and/or heterocyclic compounds and,
c) mixtures thereof.

18. The odor-controlling container of claim 18 wherein the perfume raw material is an aromatic ester.

19. A method of controlling disposal odors from soiled absorbent articles, such as diapers, sanitary napkins, panty liners, tampons, adult incontinence articles, and the like, and/or other wastes associated with a broad spectrum of odoriferous materials, such as animal bodily fluids and feces, putrefying food wastes, combustion by-products from tobacco products, and the like, any of which may or may not contain reactive functional groups by:
a) providing a sealable odor-controlling container having at least one odor-controlling composition distributed uniformly to cover the inner surface of the container walls,
b) placing into the odor-controlling container the odoriferous wastes, and
c) sealing the odor-controlling container.

20. A kit comprising a package containing one or more absorbent articles and the odor-controlling container of claim 1 for disposal of said articles when soiled.
